(19) United States
(12) Patent Application Publication
(10) Pub. No.: US 2023/0076741 A1
(43) Pub. Date: Mar. 9, 2023

(54) HANDHELD MICROSURGICAL ROBOT

(71) Applicant: KOREA INSTITUTE OF SCIENCE AND TECHNOLOGY, Seoul (KR)

(72) Inventors: Sungwook YANG, Seoul (KR); Eunchan KIM, Seoul (KR); Ingu CHOI, Seoul (KR)

(73) Assignee: KOREA INSTITUTE OF SCIENCE AND TECHNOLOGY, Seoul (KR)

(21) Appl. No.: 17/633,144

(22) PCT Filed: Oct. 19, 2021
(86) PCT No.: PCT/KR2021/014594

§ 371 Date: Feb. 4, 2022

(30) Foreign Application Priority Data
Aug. 26, 2021 (KR) 10-2021-0113160

(51) Int. Cl. A61B 34/30; A61B 34/00; A61B 5/06

(52) U.S. Cl. CPC A61B 34/30; A61B 34/72; A61B 34/75; A61B 5/065; A61B 2034/304; A61B 2034/305; A61B 2017/2902

(57) ABSTRACT

A handheld microsurgical robot according to an embodiment of the present disclosure includes a tool, and a driving mechanism configured to detachably fix the tool and operate the tool, wherein the driving mechanism includes a platform supporting the tool, a plurality of driving assemblies connected to the platform and configured to operate the platform; and a base configured to fix the plurality of driving assemblies, wherein each of the plurality of driving assemblies is capable of a linear motion with respect to the base and is capable of a rotational motion with respect to the base, wherein a position and an angle of the platform with respect to the base are controlled by the linear motion of each of the plurality of driving assemblies.

### TECHNICAL FIELD

The present disclosure relates to a handheld microsurgical robot, and more particularly, to a handheld microsurgical robot capable of correcting fine hand tremors.

### DESCRIPTION ABOUT NATIONAL RESEARCH AND DEVELOPMENT SUPPORT

This study was supported by the Ministry of Science and ICT (Project No.: 1711131233, Project Name: Development of Multi-Functional Handheld Surgical Robot for Microsurgical Treatment of Intractable Brain Tumors and System Integration) under the superintendence of the National Research Foundation of Korea.

### BACKGROUND ART

A brain tumor refers to any tumor occurring within the skull. Recently, the prevalence of intractable brain tumors has increased due to population aging, and in the case of intractable brain tumors, a survival rate is extremely low. Accordingly, treatment of increasing intractable brain tumors due to population aging has become a medical challenge to be overcome nationally.

In the brain, there is a cerebrovascular barrier that blocks the delivery of foreign substances in blood vessels to the brain, which hinders the introduction of anticancer drugs to the brain and thus the effect of chemotherapy may be limited. Accordingly, brain surgery is one of important treatments for accessible brain tumors. Because the degree of tumor resection is a decisive factor in a patient's treatment outcome and prognosis, the goal of brain surgery is to remove as much tumor tissue as possible.

Because the brain is composed of brain tissue and many neural structures that are responsible for important functions, the risk of surgery is high. In particular, when a temporary or permanent neurological disorder occurs due to brain surgery, it is directly related to a patient's quality of life. Accordingly, minimal resection for preservation of brain functions is important in brain surgery. However, when a malignant tumor is passively removed for minimal resection during brain surgery, the tumor may not be completely removed. Accordingly, the recurrence of the tumor may greatly shorten the patient's life, or may require additional treatment, and there may be risks such as complications due to the additional treatment and neurological disorders due to the recurrent tumor. Accordingly, brain tumor surgery requires “maximal safe resection” that may achieve both goals of maximizing the extent of resection while minimizing brain damage.

In this regard, there is a need for microsurgical treatment for removing only a tumor site while minimizing damage to normal tissue, and in particular, there is a need for a technology capable of micro-precise diagnosis and treatment for brain tumors.

### DISCLOSURE

### Technical Problem

An object of the present disclosure for solving the problems of the prior art is to provide a handheld microsurgical robot capable of correcting fine hand tremors, which may be used when microsurgical treatment such as brain surgery is required.

### Technical Solution

To achieve the object, a handheld microsurgical robot according to an embodiment of the present disclosure includes a tool, and a driving mechanism configured to detachably fix the tool and operate the tool, wherein the driving mechanism includes a platform supporting the tool, a plurality of driving assemblies connected to the platform and configured to operate the platform, and a base configured to fix the plurality of driving assemblies, wherein each of the plurality of driving assemblies is capable of a linear motion with respect to the base and is capable of a rotational motion with respect to the base, wherein a position and an angle of the platform with respect to the base are controlled by the linear motion of each of the plurality of driving assemblies.

Also, the driving mechanism may include six driving assemblies, wherein three degrees of freedom in position and three degrees of freedom in angle of the platform with respect to the base are controlled by the linear motion of each of the plurality of driving assemblies.

Also, each of the plurality of driving assemblies may include a motor fixed to the base, a spline shaft linearly moving due to the motor, a universal joint connected to an end of the spline shaft, and a spherical joint having an end portion connected to the universal joint and the other end portion connected to the platform.

Also, the handheld microsurgical robot may further include a detector configured to detect a pose of the tool, and a controller configured to control the motor of each of the plurality of driving assemblies.

Also, the detector may be configured to detect the pose of the tool based on positions and movement directions of three markers located on the platform.

Also, the controller may be further to control the motor of each of the plurality of driving assemblies to maintain a certain pose of the tool.

Also, the handheld microsurgical robot may further include a low-pass filter configured to remove physiological tremors of a user of the handheld microsurgical robot.

Also, the low-pass filter may have a cutoff frequency of 5 Hz or less.

Also, the low-pass filter may have a cutoff frequency of 2 Hz or less.

Also, the handheld microsurgical robot may further include a housing, a front cover coupled to a side of the housing and allowing the tool to pass therethrough, and a rear cover coupled to the other side of the housing.

Also, the handheld microsurgical robot may further include a tool connection member connected to the tool, a rear opening formed so that the tool connection member is inserted by passing through the rear cover, and a guide tube located in the housing, and configured to guide the tool connection member toward the tool.

### Advantageous Effects

A handheld microsurgical robot according to an embodiment of the present disclosure may maintain a position and an angle of a front end of a tool by removing physiological tremors of a user, and may prevent an excessive force from being applied to a surgical site by removing an unintentional movement of the user. Accordingly, the handheld microsurgical robot according to an embodiment of the present disclosure may implement microsurgical treatment for removing only a tumor site while minimizing damage to normal tissue.

Also, when a lesion in the body is imaged by using the handheld microsurgical robot according to an embodiment of the present disclosure, an optimal distance for obtaining a clear image may be maintained by removing physiological tremors of the user.

### DESCRIPTION OF DRAWINGS

FIG. 1 is a perspective view illustrating a handheld microsurgical robot according to an embodiment of the present disclosure.

FIG. 2 is an enlarged perspective view illustrating a driving mechanism of the handheld microsurgical robot of FIG. 1.

FIG. 3 is an exploded view illustrating the driving mechanism of FIG. 2.

FIG. 4 is a conceptual diagram for describing an operation of a handheld microsurgical robot according to an embodiment of the present disclosure.

FIGS. 5A to 5C are views for describing an example of adjusting a position of a tool of the handheld microsurgical robot of FIG. 1.

FIG. 6 is a view for describing another example of a position of a tool of the handheld microsurgical robot of FIG. 1.

FIG. 7 is a view for describing that a position of a tool is adjusted to diagnose a lesion by using the handheld microsurgical robot of FIG. 1.

FIG. 8 is a view for describing an example of measuring a force applied by a front end of a tool of the handheld microsurgical robot of FIG. 1.

FIG. 9 is a graph showing a force measured in FIG. 8.

[Description of Reference Numerals]

| | | | |
|---|---|---|---|
| 1: | handheld microsurgical robot | 140: | spline shaft |
| 2: | tool | 150: | spline guide |
| 10: | driving mechanism | 160: | base |
| 20: | printed circuit board | 161: | first member |
| 30: | housing | 162: | first through hole |
| 31: | front cover | 163: | connection member |
| 32: | rear cover | 165: | second member |
| 33: | rear opening | 166: | second through hole |
| 34: | guide tube | 167: | motor receiver |
| 40: | connector | 170: | coupler |
| 100: | adaptor | 171: | screw |
| 110: | platform | 180: | motor |
| 111: | marker | 181: | motor fixing portion |
| 120: | spherical joint | 130: | universal joint |

### BEST MODE

Hereinafter, various embodiments of the present disclosure will be described in detail with reference to the accompanying drawings. In adding reference numerals to elements of each drawing, it should be noted that the same elements are denoted by the same reference numerals as much as possible even when they are indicated on different drawings. Also, in describing embodiments of the present disclosure, detailed descriptions of related well-known functions or configurations that may blur the points of the embodiments of the present disclosure are omitted.

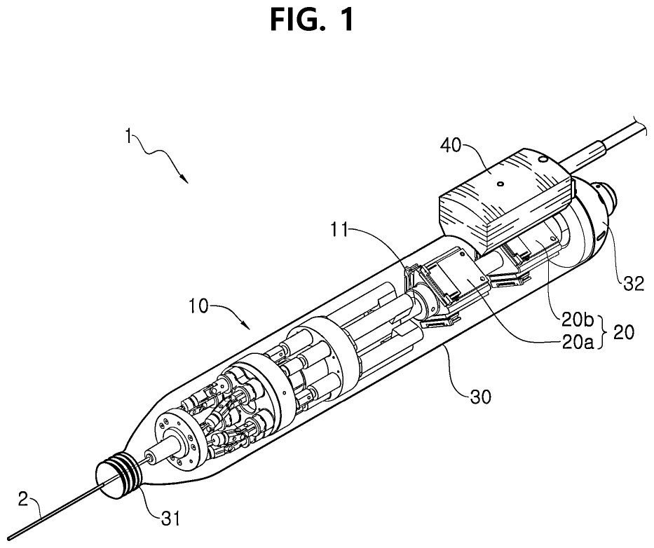

FIG. 1 is a perspective view illustrating a handheld microsurgical robot according to an embodiment of the present disclosure.

Referring to FIG. 1, a handheld microsurgical robot 1 may include a tool 2, a driving mechanism 10 for operating the tool 2, a printed circuit board 20 for controlling an operation of the driving mechanism 10, a housing 30, a front cover 31, a rear cover 32, a guide tube 34, and a connector 40.

The tool 2 is a microsurgical tool that may be inserted into the body to perform various procedures, and may be a member longitudinally extending to be inserted into the body. Examples of the tool 2 may include a cutting mechanism for removing a part of tissue in the body, a drilling mechanism for penetrating a part of body tissue, a needle for injecting a drug into the body, an endoscopic device for imaging the inside of the body, and a catheter. However, types of the tool 2 are not limited thereto, and may include various tools that may be inserted into the body to perform procedures, surgery, diagnosis, etc.

The tool 2 may be coupled to the handheld microsurgical robot 1 and may operate under the control of the handheld microsurgical robot 1. When necessary, the handheld microsurgical robot 1 may include a tool connection member (not shown) for connecting the tool 2 to the outside of the handheld microsurgical robot 1 for an operation of the tool 2.

The tool connection member is a member directly or indirectly connected to the tool 2. For example, the tool connection member may be a member such as a wire for supplying power to the tool 2 from the outside of the handheld microsurgical robot 1 or applying a signal generated from the tool 2 to the outside. In another example, the tool connection member may be a member for supplying a material introduced into the body through the tool 2 inserted into the body, or may be a member directly inserted into the body through the tool 2.

The driving mechanism 10 may operate the tool 2. To this end, the driving mechanism 10 may include a plurality of driving assemblies, and an operation of the tool 2 may be controlled by an operation of each of the driving assemblies. The driving mechanism 10 will be described below in detail.

The printed circuit board 20 may include a controller, and the controller may be, for example, a microcontroller unit (MCU). The MCU includes at least one processor. The processor may include an array of logic gates, or may include a combination of a general-purpose microprocessor and a memory in which a program executable in the microprocessor is stored. Also, it will be understood by one of ordinary skill in the art related to the present embodiment that the processor includes another type of hardware.

The MCU may be hardware for controlling an overall operation of the handheld microsurgical robot 1. For example, a controller of the MCU may perform processing for an operation of the driving mechanism 10. The printed circuit board 20 may include three first printed circuit boards 20a and three second printed circuit board 20b, and each printed circuit board may control an operation of a motor of each driving assembly of the driving mechanism 10.

In another embodiment, the controller of the MCU may maintain a pose of the tool 2 and a position of a tip of the tool 2 by controlling an operation of each driving assembly of the driving mechanism 10, based on a result of at least one parameter detected by a detector including at least one sensor.

Also, the controller may include a low-pass filter for removing physiological tremors transmitted to the handheld microsurgical robot 1 while a user uses the handheld microsurgical robot 1. The low-pass filter will be described below in detail.

The controller may execute each process through the printed circuit board 20 provided in the handheld microsurgical robot 1, or may execute each process through distributed control by cooperation with another controller (e.g., controller provided in a computer or a laptop) outside the handheld microsurgical robot 1.

The housing 30 is a member forming the exterior of the handheld microsurgical robot 1. The housing 30 may have a shape and a size suitable for the user to easily hold the handheld microsurgical robot 1.

The front cover 31 is coupled to a side of the housing 30, and the rear cover 32 is coupled to the other side of the housing 30. The tool 2 may pass through the front cover 31 and may protrude out of the handheld microsurgical robot 1. A rear opening 33 may be formed in the rear cover 32, and the tool connection member (not shown) connected to the tool 2 may pass through the rear opening 33 and may be inserted into the handheld microsurgical robot 1.

The guide tube 34 may be located in the housing 30, and may extend in parallel to an extension direction of the housing 30. For example, the guide tube 34 may be located at the center of the housing 30 and may extend, and components of the handheld microsurgical robot 1 such as the driving mechanism 10 and the printed circuit board 20 may surround the guide tube 34. The guide tube 34 may guide the tool connection member inserted into the handheld microsurgical robot 1 toward the tool 2.

The connector 40 may connect the handheld microsurgical robot 1 to an external device. For example, the connector 40 may connect the handheld microsurgical robot 1 to an external computer (not shown), and coordinate data of a platform 110 obtained from three markers 111 described below may be transmitted to the external computer through the connector 40. The external computer may detect and/or track a change in a position and an angle of the platform 110. That is, the detector may include the marker 111 located on the handheld microsurgical robot 1 to detect a pose of the tool (i.e., a position and an angle of the tool 2) and the external computer for processing position data of the platform 110 obtained from the marker 111.

The connector 40 may have not only a function of transmitting data but also a function of supplying power for operating the handheld microsurgical robot 1 when necessary. Also, the handheld microsurgical robot 1 may receive data about an operation of the handheld microsurgical robot 1 from the external computer through the connector 40.

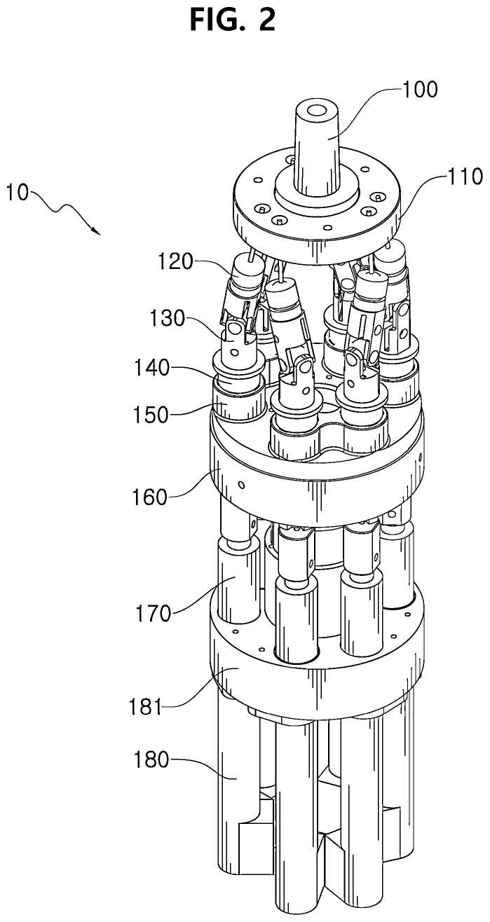

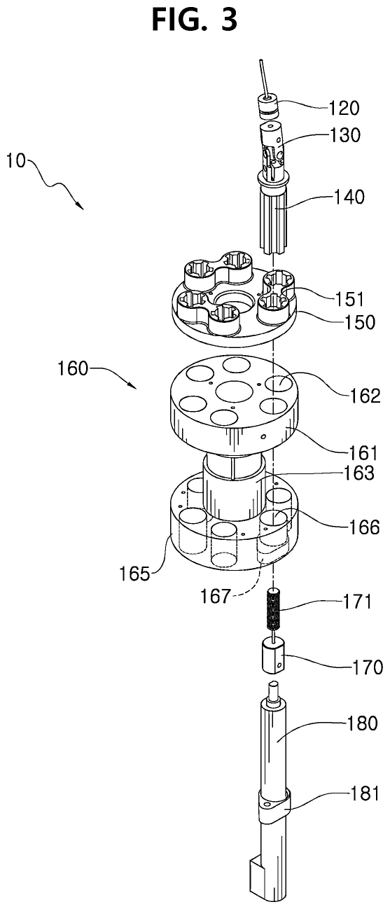

FIG. 2 is an enlarged perspective view illustrating a driving mechanism of the handheld microsurgical robot of FIG. 1. FIG. 3 is an exploded view illustrating the driving mechanism of FIG. 2.

Referring to FIGS. 2 and 3, the driving mechanism 10 for operating the tool 2 of the handheld microsurgical robot 1 may include an adaptor 100 configured to detachably fix the tool 2, the platform 110 on which the adaptor 100 is mounted, a plurality of driving assemblies 120, 130, 140, 150, 170, 180 connected to the platform 110 and configured to operate the platform 110, and a base 160 to which the plurality of driving assemblies 120, 130, 140, 150, 170, 180 are fixed.

The adapter 100 may detachably fix the tool 2. The tool 2 may be replaced with another tool 2 when necessary, and when the tool 2 is inserted into the adapter 100, the adapter 100 may firmly fix the tool 2. Accordingly, because a movement of the tool 2 such as shaking with respect to the platform 110 is limited, the tool 2 may be precisely controlled through operation control of the platform 110.

The platform 110 is a member supporting the tool 2 through the adapter 100. For example, although the platform 110 may have a circular flat plate shape as shown in FIG. 2, the platform 110 is not limited thereto and may have any of various shapes when necessary. The driving mechanism 10 is connected to a surface of the platform 110 supporting the tool 2 and the other surface in the opposite direction. The platform 110 may be moved by the driving mechanism 10 connected to the platform 110, and a movement of the platform 110 may be controlled by a controller. Because the tool 2 is supported on the platform 110 due to the adapter 100, a pose of the tool 2 (i.e., a position and an angle of the tool 2) may be controlled by the movement of the platform 110.

The marker 111 may be located on the platform 110 to detect the pose of the tool 2. That is, the controller may calculate the pose of the tool 2 by detecting a position and an angle of the platform 110. A plurality of markers 111 may be located on the platform 110, and the plurality of markers 111 may be spaced apart from one another at the same intervals. For example, as shown in FIG. 2, three markers 111 may be arranged on the platform 110 to have a virtual triangular shape. However, the number, arrangement positions, an arrangement shape, etc. of the markers 111 are not limited thereto, and may be modified in various ways to detect the position and the angle of the platform 110.

The plurality of driving assemblies 120, 130, 140, 150, 170, 180 may operate the platform 110. The plurality of driving assemblies are connected to a plurality of points of the platform 110 for a free operation of the platform 110. For example, as shown in FIG. 2, the plurality of driving assemblies may be six driving assemblies. The six driving assemblies may be arranged so that every two are adjacent to each other, and the six driving assemblies may be connected to three points on the platform 110. The three points may have a virtual equilateral triangular shape.

Referring to an embodiment of FIGS. 2 and 3, each of the plurality of driving assemblies sequentially includes a spherical joint 120, a universal joint 130, a spline shaft 140, a spline guide 150 for guiding a linear motion of the spline shaft 140, a coupler 170, and a motor 180 from top to bottom of the drawing.

The spherical joint 120 has an end portion connected to the universal joint 130 and the other end portion connected to the platform 110. The spherical joint 120 connects the platform 110 to the universal joint 130 to be freely rotatable.

The universal joint 130 has an end portion connected to the spherical joint 120 and the other end portion connected to the spline shaft 140. The universal joint 130 connects the spherical joint 120 to the spline shaft 140 even when there is a change in an angle between the spherical joint 120 and the spline shaft 140, and facilitates power transmission between the spherical joint 120 and the spline shaft 140.

The spline shaft 140 has an end portion connected to the universal joint 130. The spline shaft 140 may be inserted through a spline hole 151 formed in the spline guide 150. A shape of a spline formed on a side surface of the spline shaft 140 and a shape of the spline hole 151 of the spline guide 150 may match each other, so that a linear motion of the spline shaft 140 is guided by the spline guide 150.

The motor 180 may cause the spline shaft 140 to linearly move. For example, the coupler 170 including a screw 171 may be mounted on a driving shaft of the motor 180, and the screw 171 may be rotated by the motor 180. The screw 171 may be inserted into the spline shaft 140, and the spline shaft 140 may linearly move due to the rotation of the screw 171. That is, a rotational motion of the motor 180 may be converted into a linear motion of the spline shaft 140 through the coupler 170. However, a configuration for linearly moving the spline shaft 140 is not limited to that described above. For example, the motor 180 may be a linear motor, and the spline shaft 140 may be directly connected to the linear motor and the spline shaft 140 may be linearly moved by the linear motor.

The base 160 is a member for fixing the motor 180. For example, the base 160 may include a first member 161, a second member 165, and a connection member 163 for connecting the first member 161 to the second member 165. A first through hole 162 through which the spline shaft 140 passes may be formed in the first member 161, and a second through hole 166 through which the motor 180 passes may be formed in the second member 165. Because a driving assembly passes through the first through hole 162 and the second through hole 166, the same number of first and second through holes 162 and 166 as the plurality of driving assemblies may be formed. For example, as shown in FIG. 3, a plurality of first and second through holes 162 and 166 (6 holes in the embodiment of FIG. 3) may be formed at the same intervals in the first and second members 161 and 165.

The motor 180 may include a motor fixing portion 181 for fixing the motor 180. Also, a motor receiver 167 for accommodating the motor fixing portion 181 may be formed in the second member 165 of the base 160. A shape of the motor receiver 167 may correspond to a shape of the motor fixing portion 181. Because the motor fixing portion 181 is accommodated in the motor receiver 167 having the corresponding shape, the motor 180 may be stably fixed on the base 160.

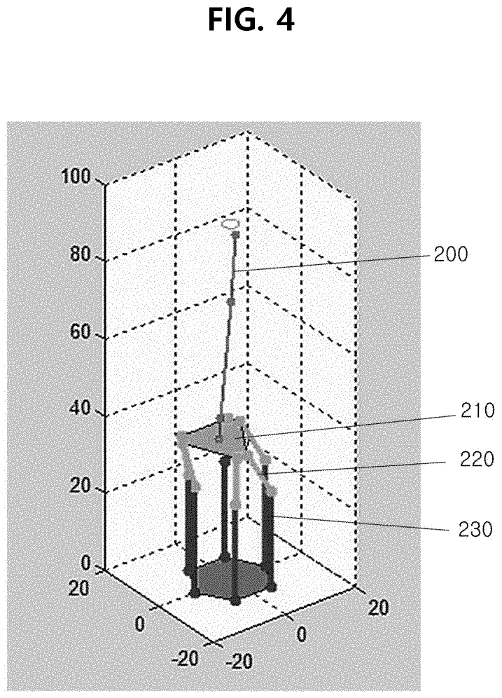

FIG. 4 is a conceptual diagram for describing an operation of a handheld microsurgical robot according to an embodiment of the present disclosure. The driving mechanism 10 is schematically illustrated in FIG. 4.

Referring to FIG. 4, a tool 200 may be supported on a platform 210, and six driving assemblies are connected to the platform 210. Each driving assembly includes a pivoting portion 220 connected to the platform 210 and a linearly moving portion 230 connected to the pivoting portion 220. The linearly moving portion 230 linearly moves with respect to a base. The pivoting portion 220 may connect the platform 210 to the linearly moving portion 230, and the pivoting portion 220 may freely pivot.

Referring to the driving assembly described with reference to FIGS. 2 and 3, the pivoting portion 220 may correspond to the spherical joint 120 and the universal joint 130, and the linearly moving portion 230 may correspond to the spline shaft 140 and the motor 180 for driving the spline shaft 140. Accordingly, each driving assembly may linearly move with respect to the base 160 and may pivot with respect to the base 160, and a position and an angle of the platform 110 with respect to the base 160 may be controlled by a linear motion of each driving assembly.

The six driving assemblies may be arranged so that every two are adjacent to each other, and the six driving assemblies may be connected to three points on the platform 210. The six driving assemblies may have a 6-prismatic-universal-spherical (PUS) kinematic structure. Three degrees of freedom in position and three degrees of freedom in angle of the platform 210 with respect to the base may be controlled by a linear motion of each driving assembly.

The 6-PUS structure has a light moving mass and small inertia because all prism joints (linearly moving portions due to the motor) are fixed to the base. Accordingly, the driving mechanism 10 of the present disclosure may provide higher dynamic performance and may prevent collision between links of each joint.

Inverse kinematics may be used to determine a pose of the tool 2. A pose of the tool 2 that is given a remote center of motion (RCM) may be calculated as a displacement of the spline shaft 140 by each motor 180 based on inverse kinematics. Accordingly, a pose of the tool 2 may be controlled based on a displacement of the spline shaft 140 by each motor 180.

For example, a detector of the handheld microsurgical robot 1 may detect a pose of the tool 2 based on positions and movement directions of three markers 111 located on the platform 110. A controller of the handheld microsurgical robot 1 may calculate a displacement of the spline shaft 140 of each driving assembly for maintaining a specific pose of the tool 2, and may control each motor 180 to operate based on a calculated displacement of each spline shaft 140.

A voluntary movement of a user in microsurgical treatment is a frequency signal of less than 2 Hz. In contrast, physiological tremors of the user generally have a root-mean-square (RMS) amplitude of about 50 μm to about 200 μm at a frequency of 6 Hz to 14 Hz. Accordingly, the controller of the handheld microsurgical robot 1 may include a low-pass filter for passing only a voluntary movement of the user by removing physiological tremors of the user. By considering frequencies related to the voluntary movement and the physiological tremors of the user, a cutoff frequency of the low-pass filter may be equal to or less than 5 Hz, and preferably, may be equal to or less than 2 Hz.

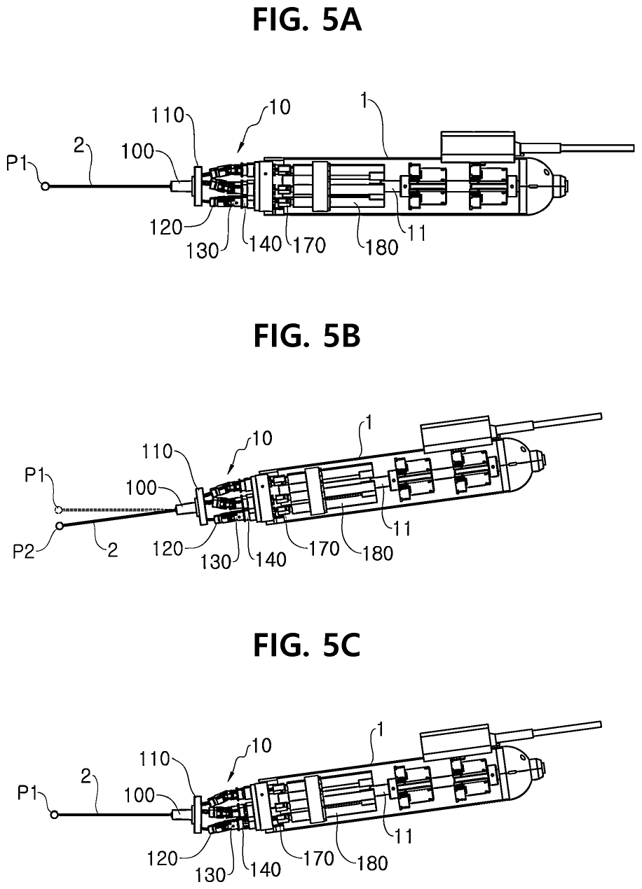

FIGS. 5A to 5C are views for describing an example of adjusting a position of a tool of the handheld microsurgical robot of FIG. 1.

Referring to FIG. 5A, a front end of the tool 2 is located at P1 when the handheld microsurgical robot 1 is used. The handheld microsurgical robot 1 illustrated in FIG. 5A is in a horizontal state.

In FIG. 5B, the handheld microsurgical robot 1 is tilted due to physiological tremors of a user when the handheld microsurgical robot 1 is used. When the handheld microsurgical robot 1 is tilted, a position of the front end of the tool 2 changes from P1 to P2.

In FIG. 5C, a position of the tool 2 of the handheld microsurgical robot 1 is adjusted. Although the handheld microsurgical robot 1 is tilted as in FIG. 5B, a position of the front end of the tool 2 is adjusted from P2 to P1 through an operation of the driving mechanism 10.

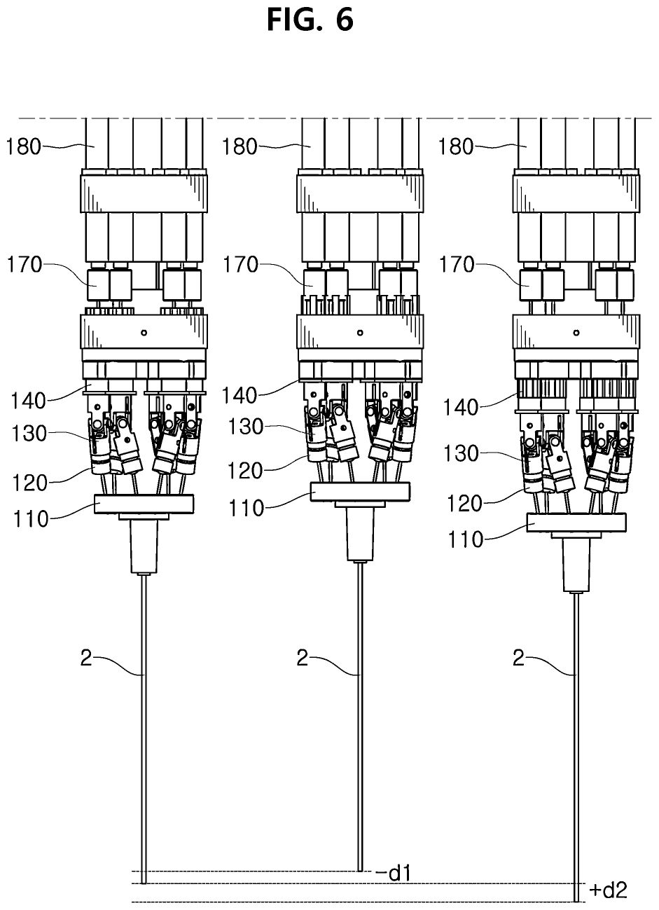

FIG. 6 is a view for describing another example of adjusting a position of a tool of the handheld microsurgical robot of FIG. 1.

Referring to FIG. 6, in FIG. 6(A), the handheld microsurgical robot 1 is located at a reference position. In FIG. 6(B), a front end of the tool 2 of the handheld microsurgical robot 1 moves by −d1 in a longitudinal direction of the tool 2. In FIG. 6(C), the front end of the tool 2 of the handheld microsurgical robot 1 moves by +d2 in the longitudinal direction of the tool 2. The tool 2 may move in the longitudinal direction through an operation of the driving mechanism 10 in a state where the handheld microsurgical robot 1 does not move. Accordingly, although the handheld microsurgical robot 1 moves in the longitudinal direction due to physiological tremors of a user when the handheld microsurgical robot 1 is used, a position of the tool 2 in the longitudinal direction may be adjusted through an operation of the driving mechanism 10.

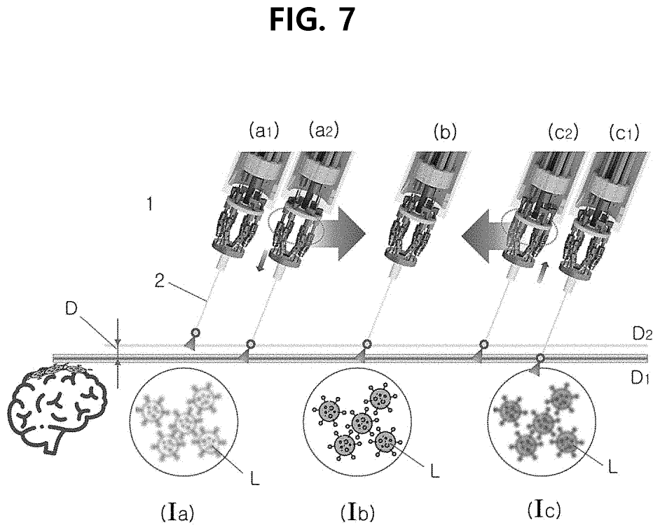

FIG. 7 is a view for describing that a position of a tool is adjusted to diagnose a lesion by using the handheld microsurgical robot of FIG. 1.

Referring to FIG. 7, the tool 2 is an endoscopic tool for imaging a lesion L in the body. In order to obtain a clear image during the imaging of the lesion L, a front end of the tool 2 should maintain a certain distance D2 spaced apart by an appropriate distance D from a distance D1 at which the lesion L is located. For example, a clear image Ib of the lesion L may be obtained when the front end of the tool 2 is located at the certain distance D2 as in (B) of FIG. 7.

Due to physiological tremors of a user during use of the handheld microsurgical robot 1, when the front end of the tool 2 at the certain distance D2 moves away from the distance D1 at which the lesion L is located as in (A1) of FIG. 7, or when the front end of the tool 2 at the certain distance D2 moves toward the distance D1 at which the lesion L is located as in (C1) of FIG. 7, an unclear image Ia or Ic of the lesion L is obtained.

Accordingly, as described with reference to FIG. 6, in the case of (A1) of FIG. 7, a position of the tool 2 in a longitudinal direction may be adjusted through an operation of the driving mechanism 10 so that the front end of the tool 2 of the handheld microsurgical robot 1 maintains the certain distance D2. That is, the platform 110 linearly moves away from the base 160, and thus the front end of the tool 2 of the handheld microsurgical robot 1 maintains the certain distance D2 as in (A2) of FIG. 7.

Also, in the case of (C1) of FIG. 7, a position of the tool 2 in the longitudinal direction may be adjusted through an operation of the driving mechanism 10 so that the front end of the tool 2 of the handheld microsurgical robot 1 maintains the certain distance D2. That is, the platform 110 linearly moves toward the base 160, and thus the front end of the tool 2 of the handheld microsurgical robot 1 maintains the certain distance D2 as in (C2) of FIG. 7.

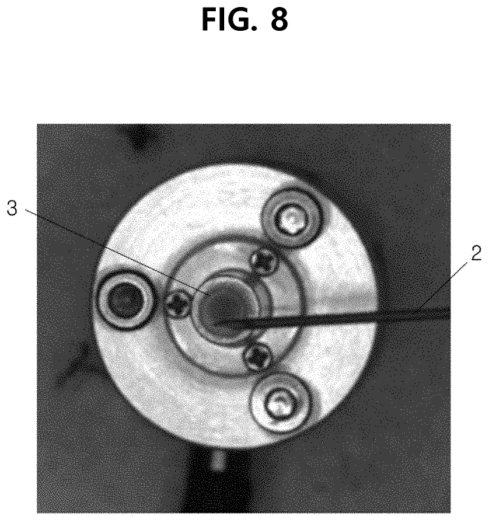

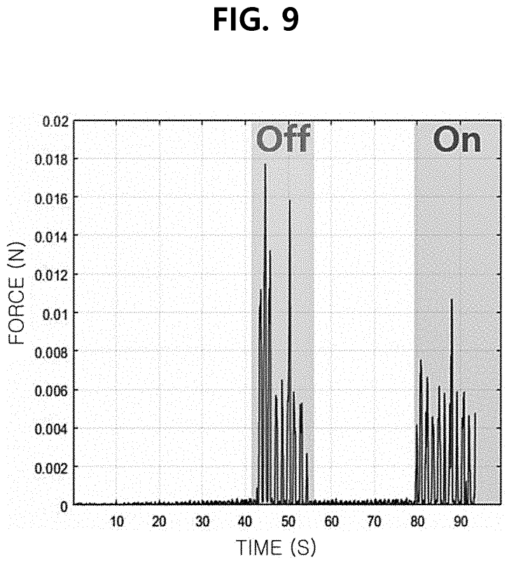

FIG. 8 is a view for describing an example of measuring a force applied by a front end of a tool of the handheld microsurgical robot of FIG. 1. FIG. 9 is a graph showing a force measured in FIG. 8.

Referring to FIG. 8, a force sensor 3 for measuring a contact force according to contact of a front end of the tool 2 is illustrated. A force applied by the front end of the tool 2 was measured by causing the front end of the tool 2 of the handheld microsurgical robot 1 to repeatedly contact a measurement portion of the force sensor 3.

FIG. 9 shows a result obtained after the front end of the tool 2 repeatedly contacted the measurement portion of the force sensor 3. First, a contact force of the front end of the tool 2 was measured in a state where the handheld microsurgical robot 1 was turned off, and then a contact force of the front end of the tool 2 was measured in a state where the handheld microsurgical robot 1 was turned on.

In a state where the handheld microsurgical robot 1 was turned off, a contact force of the tool 2 was not constant and an excessive contact force was frequently generated. That is, in a state where the handheld microsurgical robot 1 was turned off, an unintentional movement such as physiological tremors of a user occurred, which caused an excessive contact force.

In contrast, in a state where the handheld microsurgical robot 1 was turned on, a contact force of the tool 2 was kept constant and a frequency of an excessive contact force was reduced. That is, in a state where the handheld microsurgical robot 1 was turned on, an unintentional movement of the user was removed and only a voluntary movement of the user was transmitted to the tool 2. Accordingly, the handheld microsurgical robot 1 according to an embodiment of the present disclosure may implement microsurgical treatment for removing only a tumor site while minimizing damage to normal tissue.

The above description is merely illustrative of the technical idea of the present disclosure, and one of ordinary skill in the art to which the present disclosure pertains will be able to make various modifications and variations without departing from the essential characteristics of the present disclosure. Accordingly, the embodiments of the present disclosure should be considered in descriptive sense only and not for purposes of limitation of the scope of the present disclosure. The scope of the present disclosure is defined not by the detailed description of the present disclosure but by the appended claims, and all differences within the scope will be construed as being included in the present disclosure.

### INDUSTRIAL APPLICABILITY

A handheld microsurgical robot according to embodiments of the present disclosure may implement microsurgical treatment for removing only a tumor site while minimizing damage to normal tissue, and thus high industrial applicability is expected in the field of microsurgical treatment.

1. A handheld microsurgical robot (1) comprising:
a tool (2); and
a driving mechanism (10) configured to detachably fix the tool (2) and operate the tool (2),
wherein the driving mechanism (10) comprises:
a platform (110) supporting the tool (2);
a plurality of driving assemblies connected to the platform (110) and configured to operate the platform (110); and
a base (160) configured to fix the plurality of driving assemblies,
wherein each of the plurality of driving assemblies is capable of a linear motion with respect to the base (160) and is capable of a rotational motion with respect to the base (160),
wherein a position and an angle of the platform (110) with respect to the base (160) are controlled by the linear motion of each of the plurality of driving assemblies.

2. The handheld microsurgical robot of claim 1, wherein the driving mechanism (10) comprises six driving assemblies,
wherein three degrees of freedom in position and three degrees of freedom in angle of the platform (110) with respect to the base (160) are controlled by the linear motion of each of the plurality of driving assemblies.

3. The handheld microsurgical robot of claim 1, wherein each of the plurality of driving assemblies comprises:
a motor (180) fixed to the base (160);
a spline shaft (140) linearly moving due to the motor (180);
a universal joint (130) connected to an end of the spline shaft (140); and
a spherical joint (120) having an end portion connected to the universal joint (130) and the other end portion connected to the platform (110).

4. The handheld microsurgical robot of claim 3, further comprising:
a detector configured to detect a pose of the tool (2); and
a controller configured to control the motor (180) of each of the plurality of driving assemblies.

5. The handheld microsurgical robot of claim 4, wherein the detector is configured to detect the pose of the tool (2) based on positions and movement directions of three markers (111) located on the platform (110).

6. The handheld microsurgical robot of claim 4, wherein the controller is configured to control the motor (180) of each of the plurality of driving assemblies to maintain a certain pose of the tool (2).

7. The handheld microsurgical robot of claim 6, further comprising a low-pass filter configured to remove physiological tremors of a user of the handheld microsurgical robot (1).

8. The handheld microsurgical robot of claim 7, wherein the low-pass filter has a cutoff frequency of 5 Hz or less.

9. The handheld microsurgical robot of claim 8, wherein the low-pass filter has a cutoff frequency of 2 Hz or less.

10. The handheld microsurgical robot of claim 1, further comprising:
a housing (30);
a front cover (31) coupled to a side of the housing (30) and allowing the tool (2) to pass therethrough; and
a rear cover (32) coupled to the other side of the housing (30).

11. The handheld microsurgical robot of claim 10, further comprising:
a tool connection member connected to the tool (2);
a rear opening (33) formed so that the tool connection member is inserted by passing through the rear cover (32); and
a guide tube (34) located in the housing (30), and configured to guide the tool connection member toward the tool (2).
